(19) United States
(12) Patent Application Publication
(10) Pub. No.: US 2011/0033407 A1
(43) Pub. Date: Feb. 10, 2011

(54) Hair Treatment Means With Cloudberry Extract

(76) Inventors: Marcus Krueger, Ellerhoop (DE); Dieter Goddinger, Klein Nordende (DE)

Correspondence Address: Henkel Corporation, 10 Finderne Avenue, Suite B, Bridgewater, NJ, 08807, US

(21) Appl. No.: 12/904,555

(22) Filed: Oct. 14, 2010

Related U.S. Application Data
(63) Continuation of application No. PCT/EP2009/054196, filed on Apr. 8, 2009.

(30) Foreign Application Priority Data
Apr. 16, 2008 (DE) 10 2008 019 340.2

(51) Int. Cl. A61K 8/97; A61Q 5/00; A61Q 5/02; A61Q 5/10

(52) U.S. Cl. 424/70.9; 424/74; 424/70.11; 424/70.27

(57) ABSTRACT

Extract of cloudberries (Rubus chamaemorus) for use in agents for treating keratin fibers, particularly human hair, for improving the moisture in the fiber, repairing and restructuring the fibers and for protecting the fibers from oxidants and UV radiation, and for improving the color retention and color intensity when the fibers are colored hairs is disclosed.

### CROSS-REFERENCE TO RELATED APPLICATIONS

The present application is a continuation of International Patent Application No. PCT/EP2009/054196 filed 8 Apr. 2009, which claims priority to German Patent Application No. 10 2008 019 340.2 filed 16 Apr. 2008, both of which are incorporated herein by reference.

The present invention relates to cosmetic compositions comprising an extract of the fruits of Rubus chamaemorus (cloudberry) for treatment of keratin-containing fibers, as well as methods of using a cloudberry extract in cosmetic compositions for treatment of keratin-containing fibers in order to improve the moisture in keratin-containing fibers, particularly human hair, for protecting the fibers against oxidizing agents and UV radiation, for protecting the fibers from destructurization, as well as for improving the color retention and the color intensity of dyed fibers.

Nowadays, human hair is treated in a variety of ways with hair cosmetic preparations. These treatments include, for example, the cleaning of hair with shampoos, care and regeneration with rinses and tonics, as well as bleaching, dyeing and styling the hair with colorants, toners, permanent wave lotions and styling preparations.

In the context of this application, cosmetic compositions that influence a plurality of parameters of the substrate in a targeted manner are designated as “2-in-1 products”. By using a 2-in-1 product the consumer avoids the tedious multiple applications of a plurality of cosmetics.

Formulation of 2-in-1 products in combination with an oxidative treatment of keratin-containing fibers is challenging for the person skilled in the art. Reactive, oxidizing ingredients such as hydrogen peroxide, which are required for the oxidative fiber treatment, are not compatible with all cosmetic ingredients and active principles. Permanent, oxidative dyeing, as well as hair bleaching and setting for permanent waving typically occurs as an oxidative treatment in the presence of oxidizing agents such as hydrogen peroxide. Unfortunately, while the oxidizing agent produces the desired cosmetic effect, it also has a negative effect on the stability, feel and appearance of the keratin-containing fibers, in particular human hair. Hair damaged this way tends to be dull and brittle. In extreme cases the hair can even break. Therefore, there is a need to further improve hair care products and provide them with additional advantageous properties. In particular, a care complex should be made available that can also be used in combination with oxidizing agents.

A further aim is to protect the hair against aggressive oxidizing agents or UV sunlight by means of suitable ingredients. A disadvantage of strong and frequent sunlight is that the hair coloration quickly changes or fades (particularly for dyed hair). Preferably, this effect is avoided or minimized without the need for additional care products.

Accordingly, the present invention is directed towards improvements in the abovementioned side effects. These side effects with a 2-in-1 product are preferably reduced both during oxidative hair treatment and afterwards without negatively affecting the efficacy of the oxidative cosmetic composition, particularly with respect to color intensity, color fastness, lightening power or curling power. Moreover, oxidative treatment in the form of a 2-in-1 product for treating keratin-containing fibers, especially human hair, is preferably linked in one application step with the application of an effective protection of the fibers against environmental influences such as UV protection.

It has now been surprisingly found that this can be achieved to a high degree with a cosmetic composition containing an extract of the fruit of Rubus chamaemorus (hereinafter cloudberry fruit extract). In the context of this document, the name “cloudberry” refers to the fruit of Rubus chamaemorus.

Use of cloudberry seed oil (produced by super critical fluid extraction) in cosmetics for the treatment of skin and hair is known. Accordingly, a first subject matter of the present invention is a composition for treatment of keratin fibers, especially human hair, comprising an extract of the fruits of Rubus chamaemorus (cloudberry fruit extract) in a cosmetically acceptable carrier.

In a preferred embodiment, the cosmetic compositions are utilized for treatment of keratin fibers, especially human hair.

In the context of the present invention, hair treatment agents include hair dyes, bleaching agents, hair shampoos, hair conditioners, conditioning shampoos, hair sprays, hair rinses, hair tonics, hair masks, hair tonics, permanent wave fixing solutions, hair dye shampoos, hair dyes, hair setting lotions, hair setting preparations, hair styling preparations, blow dry wave lotions, mousse, hair gels, hair waxes or their combinations. According to the invention, an oxidative hair treatment refers to the action of an oxidative cosmetic comprising at least one oxidizing agent in a cosmetic carrier on the hair.

The cloudberry fruit extract is preferably used in the form of a composition comprising a cloudberry fruit extract in a cosmetic carrier, applied directly before, during or after the oxidative hair treatment. Application during or after the oxidative hair treatment is inventively preferred.

In the context of the invention, the term “directly before the oxidative hair treatment” is understood to mean an application that is followed by the oxidative hair treatment, wherein the composition comprising a cloudberry fruit extract was rinsed out of the hair beforehand or preferably was left on the hair and the hair is preferably still wet.

In the context of the invention, the term “after the oxidative hair treatment” is understood to mean an application that either follows the oxidative hair treatment, wherein the composition comprising a cloudberry fruit extract is applied onto the preferably still wet, towel dried hair after having rinsed out the hair dye or the bleaching agent or the wave lotion, or is applied onto the dried or wet hair after a plurality of hours or days after oxidative treatment. In both cases the composition according to the invention can be rinsed out again after a contact time of a few seconds up to 45 minutes or remain completely on the hair.

The action of the composition according to the invention actually develops during the oxidative hair treatment and also surprisingly continues after the inventive composition is intensively washed out.

For ‘simultaneous’ application of the oxidative cosmetic and a cosmetic containing a cloudberry fruit extract, both compositions can be optionally mixed together directly prior to the application, or put on the hair one after the other without an intermediate rinsing step. Accordingly in a preferred embodiment, the oxidative cosmetic composition additionally comprises a cloudberry fruit extract.

Cloudberry fruit extract is preferably provided in a cosmetic carrier. According to the invention, suitable cosmetic carriers include creams, emulsions, and gels, as well as surfactant-containing foaming solutions such as shampoos, foam aerosols or other preparations that are particularly suitable for use on the hair.

An aqueous cosmetic carrier comprises at least 50 wt. %, preferably at least 60 wt. % water.

In the context of the present invention, aqueous alcoholic cosmetic carriers refer to aqueous solutions comprising 3 to 70% by weight of a C₁-C₆ alcohol, particularly ethanol or propanol, isopropanol, butanol, isobutanol, tert.-butanol, n-pentanol, iso-pentanol, n-hexanol, iso-hexanol, glycol, glycerin, 1,2-pentane diol, 1,5-pentane diol, 1,2-hexane diol or 1,6-hexane diol. Compositions according to the invention can further comprise organic solvents such as methoxybutanol, benzyl alcohol, ethyl diglycol or 1,2-propylene glycol. Preference here is given to all water-soluble organic solvents. An inventively preferred employable cloudberry fruit extract is sold under the trade name Fruitliquid Cloudberry® by Crodarom (INCI name (EU): Glycerin, Aqua, Rubus chamaemorus Fruit Extract in Glycerin/Water).

Inventive cosmetic compositions contain cloudberry fruit extract in a quantity of 0.01 to 5.0 wt. %, preferably 0.05 to 4.0 wt. % and particularly 0.1 to 3.0 wt. %, based on total weight of the composition.

In a first preferred embodiment, the inventive compositions further comprise at least one additional active principle. This additional active principle can be chosen from non-surface active betaines, ubiquinone, ectoine, pantolactone, pantothenic acid, nicotinic acid, their derivatives and physiologically acceptable salts.

In particular, this additional active principle is chosen from L-carnitine tartrate, coenzyme Q-10, ectoine, pantolactone, pantothenic acid, taurine, panthenol, nicotinamide and their derivatives or physiologically acceptable salts. In the context of this document, the term “derivative” refers to an easily accessible, structurally closely related derivative of the starting material that can be synthesized by a single reaction step. A “derivatization” of this kind can be made enzymatically, although it is typically created by suitable synthetic transformations, usually on a functional group. Exemplary derivatives include the reaction products from the derivatizations of hydroxy, thiol, amino, carbonyl, carboxyl or carboxamide groups. Preferred derivatization methods include oxidation, reduction, alkylation, acylation, esterification, saponification, (de)protonation (salt formation with physiologically acceptable acids or bases) and peptide formation.

Particularly preferred compositions according to the invention include those comprising the cloudberry fruit extract and the additional active principle in a weight ratio range of 1:30 to 50:1, preferably in the range of 1: 10 to 30: 1 and in particular in the range of 1:5 to 10:1.

A particularly preferred group of ingredients in cosmetic compositions according to the invention are betaines. In particular, betaines according to the invention correspond to formula (A-I)—

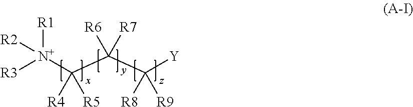

Exemplary and particularly preferred betaines of formula (A-I) include carnitine, carnitine tartrate, carnitine magnesium citrate, acetyl carnitine, 3-O-lauroyl-L-carnitine hydrochloride, S-O-octanoyl-L-carnitine hydrochloride, 3-O-palnnitoyl-L-carnitine hydrochloride, taurine, taurine lysylate, taurine tartrate, taurine ornithate, lysyl taurine and ornithyl taurine, betalaine, 1,1-dimethyl-proline, choline, choline chloride, choline bitartrate, choline dihydrogen citrate and N,N,N-trimethylglycine that is cited in the literature as a betaine compound.

Carnitine, taurine, histidine, choline, and betaine as well as their derivatives are preferably used. In this regard, compositions according to the invention can have both a compound according to formula (A-I) as well as a plurality, especially two, of compounds according to formula (A-I). In a particularly preferred embodiment of the invention, L-carnitine tartrate is employed as the active principle.

In another preferred embodiment, the cosmetic comprises taurine and/or at least one of its derivatives in addition to the cloudberry fruit extract. In the present invention, taurine refers to 2-aminoethane sulfonic acid. Preferred taurine derivatives are N-monomethyltaurine and N,N-dimethyltaurine. Further taurine derivatives in the context of the present invention include taurocholic acids and hypotaurine.

In a particularly preferred embodiment of the invention, taurine is employed as the active principle in the composition according to the invention.

In another inventively preferred embodiment, the inventive compositions comprise bioquinones that act as coenzymes. Suitable bioquinones in the inventive agents include one or more ubiquinones and/or plastoquinones. Preferred ubiquinones according to the invention have the following formula—

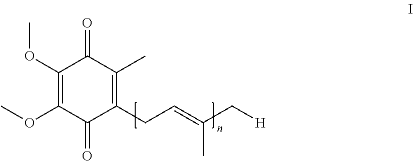

wherein n=6, 7, 8, 9 or 10. In a particularly preferred embodiment of the present invention, the cosmetic composition comprises a combination of coenzyme Q-10 (ubiquinone-50) and/or at least one of its derivatives and cranberry extract.

In another preferred embodiment of the present invention, the cosmetic composition comprises a combination of ectoine ((S)-2-methyl-1,4,5,6-tetrahydro-4-pyrimidine carboxylic acid) and/or at least one of its derivatives and cloudberry fruit extract.

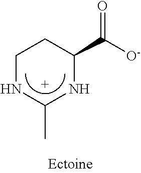

In another preferred embodiment of the present invention, the cosmetic composition comprises a combination of cloudberry fruit extract and pantolactone ((R)-dihydro-3-hydroxy-4,4-dimethyl-2(3H)-furanone).

Quite particularly preferred compositions in the context of the present invention comprise pantolactone.

In another preferred embodiment of the present invention, the cosmetic composition comprises a combination of pantothenic acid and/or at least one of its derivatives and cloudberry fruit extract. In the present invention, pantothenic acid is preferably employed as a derivative in the form of the stabilized calcium salt and sodium salt (Ca pantothenate, Na pantothenate).

In the context of the present invention, additional suitable derivatives of pantothenic acid are panthenol and pantetheine.

In another preferred embodiment of the present invention, the cosmetic composition comprises a combination of nicotinic acid and/or nicotinamide and/or at least one of the derivatives of these compounds with cloudberry fruit extract.

The term “niacin” also includes nicotinic acid and nicotinamide. Both compounds possess the same vitamin activity and can be converted into one another by the organism.

Compositions comprising, based on total weight of the composition, 0.00001 to 10.0 wt. %, preferably 0.0001 to 5.0 wt. % and particularly 0.001 to 3 wt. % of active principles from carnitine tartrate, coenzyme Q-10, ectoine, pantolactone, pantothenic acid, taurine, panthenol, nicotinamide and their derivatives or physiologically acceptable salts are particularly preferred.

Compositions according to the invention can also include carnitine tartrate, coenzyme Q-10, ectoine, pantolactone, pantothenic acid, taurine, panthenol, nicotinamide and/or their derivatives in the form of natural extracts that were extracted from natural substances containing the corresponding active principles or their derivatives.

It is further preferred that compositions according to the invention also contain at least one conditioning agent for the skin and/or the hair. Preferred conditioning agents include cationic surfactants, cationic polymers, silicones and/or vegetal oils.

Inventive compositions preferably include cationic surfactants (Tkat) in amounts of 0.05 to 10 wt. %, based on total composition. Quantities of 0.1 to 5 wt. % are particularly preferred.

Cationic polymers can also be preferably used in compositions according to the invention. Preferred cationic and commercially available polymers include—
quaternized cellulose derivatives, commercially available under the names Celquat® and Polymer JR®. The compounds Celquat® H 100, Celquat® L 200 and Polymer JR®400 are preferred quaternized cellulose derivatives.
cationic alkyl polyglycosides.
cationized honey such as the commercial product Honeyquat® 50.
cationic guar derivatives such as those products marketed under the trade names Cosmedia® Guar and Jaguar®.
polymeric dimethyldiallylammonium salts and their copolymers with esters and amides of acrylic acid and methacrylic acid. The commercially available products Merquat®100 (poly(dimethyldiallylammonium chloride)), Merquat®550 (dimethyldiallylammonium chloride-acrylamide copolymer), and Merquat® Plus 3300 (terpolymer of dimethyldiallylammonium chloride, sodium acrylate and acrylamide) are examples of such cationic polymers.
copolymers of vinyl pyrrolidone with quaternized derivatives of dialkylaminoalkyl acrylate and dialkylaminoalkyl methacrylate such as vinyl pyrrolidone-dimethylaminoethyl methacrylate copolymers quaternized with diethyl sulfate. Compounds of this type are commercially available under the trade names Gafquat® HS 100, Gaffix® VC 713, Gafquat® 734 and Gafquat® 755, with Gafquat® 734 being particularly preferred.
vinyl pyrrolidone-vinyl imidazolium methochloride copolymers, as are offered under the trade names Luviquat® FC 370, FC 550, FC 905 and HM 552.
quaternized polyvinyl alcohol.
polymers containing quaternary nitrogen atoms in the main polymer chain, known under the names Polyquaternium 2, Polyquaternium 17, Polyquaternium 18 and Polyquaternium 27.
vinyl pyrrolidone-vinyl caprolactam-acrylate terpolymers with acrylic acid esters and acrylamides as the third monomer moieties, commercially available, for example, under the trade name Aquaflex® SF 40.

Compositions according to the invention preferably contain cationic polymers in amounts of 0.01 to 10 wt. %, based on total weight of the composition. Quantities of 0.05 to 5 wt. % are preferred and quantities of 0.1 to 3 wt. % are quite particularly preferred.

Another substance group that can be additionally used as a conditioning agent in the compositions according to the invention, are the silicone oils (hereinafter also called silicones). The addition of cyclic dimethicones, designated by INCI as Cyclomethicones, is also inventively preferred. Here, cosmetic or dermatological preparations according to the invention having at least one silicone of formula (Si-4) are preferred—

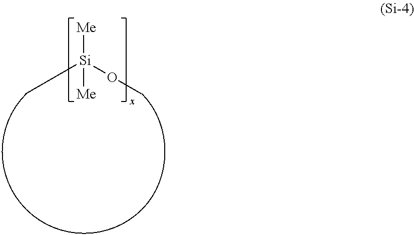

wherein x is a number from 3 to 200, advantageously from 3 to 10, more preferably from 3 to 7 and especially 3, 4, 5 or 6.

In a particularly preferred embodiment of the present invention, the conditioning agent is a conditioning silicone having a viscosity of 20,000 to 120,000 mPas, quite particularly preferably 40,000 to 80,000 mPas.

The conditioning silicone is preferably chosen from dimethicones, amodimethicones or dimethiconols.

Finally, in addition to the cloudberry fruit extract and the additional active principle as the conditioning agent, vegetal oil can also be preferably employed in the compositions according to the invention. Exemplary natural oils include amaranth seed oil, apricot stone oil, argan oil, avocado oil, babassu oil, cotton seed oil, borage seed oil, camelina oil, safflower oil, peanut oil, pomegranate stone oil, grapefruit seed oil, hemp oil, rosebush oil, hazelnut oil, elderberry seed oil, current seed oil, jojoba oil, cocoa butter, linseed oil, macadamia nut oil, maize seed oil, almond oil, marula oil, evening primrose oil, olive oil, palm oil, peach stone oil, rape seed oil, rice oil, Hippophae Rhamnoides oil, sea buckthorn seed oil, sesame oil, shea butter, soya oil, sunflower oil, grape seed oil, walnut oil or wild rose oil.

The amount of vegetal oils in the inventive compositions, based on total weight of the composition, is 0.001 to 10 wt. %, preferably 0.005 to 7 wt. %, particularly preferably 0.01 to 5 wt. % and particularly 0.05 to 3 wt. %.

It can be inventively preferred to use the composition according to the invention in the context of a color change of the hair. Here, oxidative color change is particularly preferred as the beneficial effect of the inventive composition is excellent even in the presence of an oxidizing agent. Therefore, preferred compositions according to the invention comprise at least one oxidizing agent.

In the context of the invention, oxidizing agents differ from atmospheric oxygen and possess an oxidation potential that enables them to couple disulfide bridges inside or between the proteins of the hair keratin, to oxidatively lighten the natural color pigment melanin, and/or to oxidize an oxidation dye precursor of the developer type. Oxidizing agents preferably include hydrogen peroxide and/or at least one addition product thereof, particularly on inorganic or organic compounds such as sodium perborate, sodium percarbonate, magnesium percarbonate, sodium percarbamide, polyvinyl pyrrolidone H₂O₂ (n is a positive integer greater than 0), urea peroxide and melamine peroxide.

The oxidizing cosmetic composition preferably includes the oxidizing agent in an amount of 1.0 wt. % to 10 wt. %, particularly 3.0 wt. % to 10.0 wt. %, each based on the weight of the ready for use composition.

For the purpose of color change the cosmetic compositions preferably additionally comprise at least one color-changing component. According to the object, inter alia the fastness of the obtained dyeing against environmental influences is improved.

The color-changing component is preferably chosen from—
(a) at least one oxidation dye precursor of the developer component type and optionally from at least one coupler component, and/or
(b) oxo dye precursors, and/or
(c) at least one substantive dye, and/or
(d) at least one precursor of nature-analogous dyes, and/or
(e) at least one bleach booster.

Accordingly, in a particularly preferred embodiment of the present invention, the cosmetic composition additionally comprises at least one oxidation dye precursor of the developer component type.

Usually, primary aromatic amines with an additional free or substituted hydroxyl or amino group in the para- or ortho-position, diaminopyridine derivatives, heterocyclic hydrazones, 4-aminopyrazolone derivatives as well as 2,4,5,6-tetraaminopyrimidine and derivatives thereof are employed as the developer components.

In general, m-phenylenediamine derivatives, naphthols, resorcinol and resorcinol derivatives, pyrazolones and m-aminophenol derivatives, as well as heterocyclic compounds are generally used as coupling components.

Cosmetic compositions according to the invention preferably contain developer components in an amount of 0.005 to 10% by weight, preferably 0.1 to 5% by weight, based on total weight of the composition.

Cosmetic compositions according to the invention preferably comprise coupling components in an amount of 0.005 to 10% by weight, preferably 0.1 to 5% by weight, based on total weight of the composition.

Compositions according to the invention can include as the color-changing component in the form of oxo dye precursor at least one combination of at least one compound from the following components—
1. compounds that comprise a reactive carbonyl group with at least one compound from the components; and/or
2. compounds chosen from (a) CH-acidic compounds, (b) compounds containing primary or secondary amino groups or hydroxyl groups, chosen from primary or secondary aromatic amines, nitrogen-containing heterocyclic compounds and aromatic hydroxy compounds.

The actual oxidative dye is prepared directly prior to use by mixing the separately stored dye precursor and the oxidizing agent. Accordingly, in a preferred embodiment, the cosmetic composition is mixed prior to application from a composition comprising at least one color-changing component in a cosmetic carrier, and a further composition comprising at least one oxidizing agent in a cosmetic carrier.

When using oxidizing agents, the “ready for use” preparation is expediently prepared directly prior to use by mixing a preparation containing the oxidizing agent with the preparation containing the color-changing components. The pH of the resulting “ready for use” hair preparation should be in the range 6 to 12, especially 7.5 to 10.

For color change by lightening or bleaching the hair, at least one bleach booster in addition to the oxidizing agents is preferably additionally added to the inventive cosmetic composition. Bleach boosters are preferably peroxy compounds. Inventive bleach boosting peroxy compounds include neither hydrogen peroxide addition products with other components nor hydrogen peroxide itself. Further, the choice of peroxy compound is not limited. Preferred peroxy compounds include peroxydisulfate salts, persulfate salts, (especially ammonium peroxydisulfate, potassium peroxydisulfate, sodium peroxydisulfate, ammonium persulfate, potassium persulfate, sodium persulfate, potassium peroxydiphosphate) and peroxides (such as barium peroxide and magnesium peroxide). According to the invention, inorganic compounds are preferred among these peroxide compounds and can also be employed in combination. Peroxydisulfates, particularly ammonium peroxydisulfate, are particularly preferred. Bleach boosters can be present in cosmetic compositions according to the invention in amounts of 5 to 30% by weight, particularly 8 to 20% by weight. Cosmetic compositions of the invention include, when they act as the bleaching agent, as the preferred alkalizing agent at least one compound chosen from hydroxides, carbonates, hydrogen carbonates, hydroxycarbonates, metasilicates and carbamides of ammonium, alkali metal and alkaline earth metal, and also alkali metal phosphates.

The inventive cosmetic compositions can furthermore comprise all active substances, additives and auxiliaries known for the relevant preparation.

There is no need to subsequently wash the hair with a shampoo if a strong surfactant-containing carrier was used.

In many cases the compositions include at least one surfactant, wherein not only anionic, but also zwitterionic, ampholytic, non-ionic and cationic surfactants are suitable. Choice of the surface-active substances depends on the type of composition. In the case of shampoos, at least one surfactant is chosen from anionic, zwitterionic or non-ionic surfactants. Preferably at least one anionic and at least one zwitterionic surface-active substance are included. These surfactants are preferably particularly mild surfactants. However, in many cases it has proved advantageous to include surfactants from among anionic, zwitterionic or non-ionic surfactants. Here the ratio between anionic and zwitterionic surfactants is from 10:1 to 1:5, with the ratio 5:1 to 1:2 particularly preferred. If the composition is a hair conditioning preparation, then cationic and/or non-ionic surfactants are preferably selected as the surface-active substance. Here as well, a selection of the so-called mild surfactant is once again particularly preferred.

Suitable anionic surfactants (Tanion) for the inventive preparations include all anionic surfactants that are suitable for use on the human body. Preferred anionic surfactants include alkyl sulfates, alkyl polyglycol ether sulfates and ether carboxylic acids with 10 to 18 C atoms in the alkyl group and up to 12 glycol ether groups in the molecule, sulfosuccinic acid mono and dialkyl esters with 8 to 18 C atoms in the alkyl group and sulfosuccinic acid mono-alkyl polyoxyethyl esters with 8 to 18 C atoms in the alkyl group and 1 to 6 oxyethylene groups, monoglycerin disulfates, alkyl- and alkenyl ether phosphates as well as albumin fatty acid condensates.

Particularly suitable zwitterionic surfactants include betaines such as N-alkyl-N,N-dimethylammonium glycinates (e.g., cocoalkyldimethylammonium glycinate), N-acylaminopropyl-N,N-dimethylammonium glycinates (e.g., cocoacylaminopropyldimethylammonium glycinate), and 2-alkyl-3-carboxymethyl-3-hydroxyethyl imidazolines with 8 to 18 carbon atoms in each of the alkyl or acyl groups, as well as cocoa acylaminoethyl hydroxyethyl carboxymethyl glycinate. A preferred zwitterionic surfactant is the fatty acid amide derivative known under the INCI name Cocamidopropyl Betaine.

Suitable ampholytic surfactants include N-alkyl glycines, N-alkylamino propionic acids, N-alkylamino butyric acids, N-alkylimino dipropionic acids, N-hydroxyethyl-N-alkylamidopropylglycine, N-alkyl taurines, N-alkyl sarcosines, 2-alkylamino propionic acids and alkylamino acetic acids, each with about 8 to 24 carbon atoms in the alkyl group. Typical examples of amphoteric or zwitterionic surfactants are alkyl betaines, alkylamido betaines, amino propionates, amino glycinates, imidazolinium betaines and sulfo betaines. Particularly preferred ampholytic surfactants are N-cocoa alkylamino propionate, cocoa acylaminoethylamino propionate and C₁₂-C₁₈ acyl sarcosine.

Alkylene oxide addition products to saturated, linear fatty alcohols and fatty acids, each with 2 to 30 moles ethylene oxide per mole fatty alcohol or fatty acid, are examples of preferred non-ionic surfactants. Preparations with excellent properties are also obtained when they comprise fatty acid esters of ethoxylated glycerin as the non-ionic surfactant.

The surfactants (T) can be used in quantities of 0.05 to 45 wt. %, preferably 0.1 to 30 wt. % and quite particularly preferably from 0.5 to 25 wt. %, based on total weight of the composition. Exemplary inventively usable emulsifiers include—
addition products of 4 to 30 moles ethylene oxide and/or 0 to 5 moles propylene oxide to linear fatty alcohols containing 8 to 22 carbon atoms, to fatty acids containing 12 to 22 carbon atoms and to alkyl phenols containing 8 to 15 carbon atoms in the alkyl group;
C-₁₂-C₂₂ fatty acid mono- and diesters of addition products of 1 to 30 moles ethylene oxide on polyols containing 3 to 6 carbon atoms, especially on glycerin;
ethylene oxide and polyglycerine addition products on methyl glucoside fatty acid esters, fatty acid alkanolamides and fatty acid glucamides;
C₈-C₂₂ alkyl mono and oligoglycosides and their ethoxylated analogs, wherein the degrees of oligomerization are 1.1 to 5, particularly 1.2 to 2.0, and glucose as the sugar component are preferred;
mixtures of alkyl (oligo)glucosides and fatty alcohols, for example the commercial product Montanov® 68;
partial esters of polyols containing 3-6 carbon atoms with saturated fatty acids containing 8 to 22 carbon atoms;
sterols. Sterols are understood to mean a group of steroids, which carry a hydroxyl group on carbon atom 3 of the steroid skeleton and are isolated from both animal tissue (zoosterols) and vegetal fats (phytosterols). Zoosterols include cholesterol and lanosterol. Suitable phytosterols include ergosterol, stigmasterol and sitosterol. Sterols, the so-called mycosterols, are also isolated from fungi and yeasts.
phospholipids. Among these are principally meant the glucose-phospholipids, obtainable, for example, as lecithins or phosphatidyl cholines from, for example, egg yolk or plant seeds (e.g., soya beans).
fatty acid esters of sugars and sugar alcohols such as sorbitol;
polyglycerines and polyglycerin derivatives such as polyglycerin poly-12-hydroxystearate (commercial product Dehymuls® PGPH);
linear and branched fatty acids with 8 to 30 carbon atoms and their Na, K, ammonium, Ca, Mg and Zn salts.

The inventive compositions preferably comprise emulsifiers in quantities of 0.1 to 25 wt. %, particularly 0.5 to 15 wt. %, based on total weight of the composition.

Just like cationic polymers, amphoteric polymers are likewise inventively quite particularly preferred polymers. The term amphoteric polymers embraces not only those polymers whose molecule includes both free amino groups and free —COOH or SO₃H groups and which are capable of forming inner salts, but also zwitterionic polymers whose molecule comprises quaternary ammonium groups and —COO⁻ or —SO₃⁻ groups, and polymers comprising —COOH or SO₃H groups and quaternary ammonium groups.

Compositions according to the invention preferably comprise amphoteric polymers in amounts of 0.05 to 10 wt. %, based on total weight of the composition. Quantities of 0.1 to 5 wt. % are particularly preferred.

Anionic polymers include anionic polymers having carboxylate and/or sulfonate groups. Exemplary anionic monomers from which such polymers can be made include acrylic acid, methacrylic acid, crotonic acid, maleic anhydride and 2-acrylamido-2-methylpropane sulfonic acid. Here, the acidic groups may be fully or partially present as sodium, potassium, ammonium, mono- or triethanolammonium salts. Preferred monomers include 2-acrylamido-2-methylpropane sulfonic acid and acrylic acid. Anionic polymers containing 2-acrylamido-2-methylpropane sulfonic acid alone or as the comonomer have proven to be quite particularly effective. The sulfonic acid group may be fully or partially present as the sodium, potassium, ammonium, mono- or triethanolammonium salt.

The homopolymer of 2-acrylamido-2-methylpropane sulfonic acid (commercially available, for example, under the trade name Rheothik® 11-80) is particularly preferred. Compositions according to the invention preferably comprise anionic polymers in quantities of 0.05 to 10 wt. %, based on total composition. Quantities of 0.1 to 5 wt. % are particularly preferred.

In a further embodiment, inventive compositions contain non-ionic polymers. Suitable non-ionic polymers include—
vinyl pyrrolidone-vinyl ester copolymers such as those marketed by BASF under the trade name Luviskol®, Luviskol® VA 64 and Luviskol® VA 73, each vinyl pyrrolidone-vinyl acetate copolymers, are likewise preferred non-ionic polymers.
Cellulose ethers, such as hydroxypropyl cellulose, hydroxyethyl cellulose, and methyl hydroxypropyl cellulose, as marketed for example under the trademarks Culminal® and Benecel® (AQUALON) and Natrosol® types (Hercules).
Starch and its derivatives, especially starch ethers, for example, Structure® XL (National Starch), a multifunctional, salt tolerant starch.
Shellac.
Polyvinyl pyrrolidones such as are marketed under the designation Luviskol® (BASF).

Compositions according to the invention preferably comprise non-ionic polymers in quantities of 0.05 to 10 wt. %, based on total composition. Quantities of 0.1 to 5 wt. % are particularly preferred.

According to the invention, it is also possible for the preparations to comprise a plurality, particularly two different polymers of the same charge and/or an anionic and an amphoteric and/or non-ionic polymer.

The inventive compositions preferably include polymers (P) in amounts of 0.01 to 30 wt. %, based on total composition. Quantities of 0.01 to 25 wt. %, particularly 0.01 to 15 wt. %, are particularly preferred. Compositions according to the invention particularly preferably comprise fats (D) as additional active principle. Fats (D) include fatty acids, fatty alcohols, natural and synthetic waxes that can exist both in solid form as well as liquid in aqueous dispersion, and natural and synthetic cosmetic oil components. Linear and/or branched, saturated and/or unsaturated fatty acids containing 6-30 carbon atoms can be used as the fatty acids (D1). Fatty acids containing 10 to 22 carbon atoms are preferred. Among these may be cited the isostearic acids such as the commercial products Emersol® 871 and Emersol® 875, and isopalmitic acids such as the commercial product Edenor® IP 95, as well as other fatty acids commercialized under the trade names Edenor® (Cognis). Further typical examples of such fatty acids include caproic acid, caprylic acid, 2-ethylhexanoic acid, capric acid, lauric acid, isotridecanoic acid, myristic acid, palmitic acid, palmitoleic acid, stearic acid, isostearic acid, oleic acid, elaidic acid, petroselic acid, linoleic acid, linolenic acid, elaeostearic acid, arachidonic acid, gadoleic acid, behenic acid and erucic acid as well as their industrial mixtures. Usually, the fatty acid fractions obtainable from coconut oil and palm oil are particularly preferred; in general, the addition of stearic acid is particularly preferred.

The added quantity of fatty acids ranges from 0.1-15 wt. %, based on total composition. The quantity preferably ranges from 0.5-10 wt. %, wherein quantities of 1-5 wt. % can be quite particularly advantageous.

Saturated, mono or polyunsaturated, branched or linear fatty alcohols containing C₆ to C₃₀, preferably C₁₀ to C₂₂ and quite particularly preferably C₁₂ to C₂₂ carbon atoms can be added as fatty alcohols (D2). In the scope of the invention, decanol, octanol, octenol, dodecenol, decenol, octadienol, dodecadienol, decadienol, oleyl alcohol, eruca alcohol, ricinyl alcohol, stearyl alcohol, isostearyl alcohol, cetyl alcohol, lauryl alcohol, myristyl alcohol, arachidyl alcohol, capryl alcohol, caprinic alcohol, linoleyl alcohol, linolenyl alcohol and behenyl alcohol, as well as Guerbet alcohols can be added, this listing being intended as exemplary and not limiting in character. However, fatty alcohols are preferably derived from naturally occurring fatty acids, usually obtained by reducing the fatty acid esters. Likewise, according to the invention, fatty alcohols can be added that are obtained by reducing naturally occurring triglycerides like beef tallow, palm oil, peanut oil, oil of rapeseed, cotton seed oil, soya oil, sunflower oil and linen oil, or the fatty acid esters produced from their transesterification products with appropriate alcohols, thereby producing a mixture of different fatty alcohols. Such substances can be bought, for example, under the trade names Stenol®, e.g. Stenol® 1618 or Lanette®, e.g. Lanette® O or Lorol®, e.g. Lorol® C8, Lorol® C14, Lorol® C18, Lorol® C8-18, HD-Ocenol®, Crodacol®, e.g. Crodacol® CS, Novol®, Eutanol® G, Guerbitol® 16, Guerbitol® 18, Guerbitol® 20, Isofol® 12, Isofol® 16, Isofol® 24, Isofol® 36, Isocarb® 12, Isocarb® 16 or Isocarb® 24. Wool wax alcohols such as those commercially available under the trade names Corona®, White Swan®, Coronet® or Fluilan® can also be added according to the invention. The fatty alcohols are added in quantities of 0.1-30 wt. %, based on the total preparation, preferably in quantities of 0.1-20 wt. %.

According to the invention, solid paraffins or isoparaffins, carnauba wax, bean wax, candelilla wax, ozocerite, ceresin, sperm wax, sunflower wax, fruit waxes such as apple wax or citrus wax, microwaxes of PE or PP can be added as the natural or synthetic waxes (D3). These types of waxes are available, for example, from Kahl & Co., Trittau. The added quantities are 0.1 to 50 wt. %, preferably 0.1 to 20 wt. %, and particularly preferably 0.1 to 15 wt. %, based on total composition.

Natural and synthetic cosmetic oil bodies (D4) include—
liquid paraffin oils, isoparaffin oils and synthetic hydrocarbons as well as di-n-alkyl ethers containing a total of 12 to 36 carbon atoms, particularly 12 to 24 carbon atoms such as, for example, di-n-octyl ether, di-n-decyl ether, di-n-nonyl ether, di-n-undecyl ether, di-n-dodecyl ether, n-hexyl n-octyl ether, n-octyl n-decyl ether, n-decyl n-undecyl ether, n-undecyl n-dodecyl ether and n-hexyl n-undecyl ether and di-tert.butyl ether, diisopentyl ether, di-3-ethyldecyl ether, tert.butyl n-octyl ether, isopentyl n-octyl ether and 2-methylpentyl n-octyl ether. The commercial products 1,3-di-(2-ethylhexyl)cyclohexane (Cetiol® S) and di-n-octyl ether (Cetiol® OE) can be preferred.
ester oils. Ester oils are understood to mean the esters of C₆-C₃₀ fatty acids with C₂-C₃₀ fatty alcohols. Monoesters of fatty acids with alcohols having 2 to 24 carbon atoms are preferred. Examples of the fatty acids moieties in the esters are caproic acid, caprylic acid, 2-ethylhexanoic acid, capric acid, lauric acid, isotridecanoic acid, myristic acid, palmitic acid, palmitoleic acid, stearic acid, isostearic acid, oleic acid, elaidic acid, petroselic acid, linoleic acid, linolenic acid, elaeostearic acid, arachidonic acid, gadoleic acid, behenic acid and erucic acid as well as their industrial mixtures. Examples of the fatty alcohol moieties in the ester oils are isopropyl alcohol, capronyl alcohol, capryl alcohol, 2-ethylhexyl alcohol, capric alcohol, lauryl alcohol, isotridecyl alcohol, myristyl alcohol, cetyl alcohol, palmoleyl alcohol, stearyl alcohol, isostearyl alcohol, oleyl alcohol, elaidyl alcohol, petroselinyl alcohol, linoleyl alcohol, linolenyl alcohol, elaeostearyl alcohol, arachyl alcohol, gadoleyl alcohol, behenyl alcohol, erucyl alcohol and brassidyl alcohol as well as their industrial mixtures. isopropyl myristate (Rilanit® IPM), isononanoic acid-C₁₆-₁₈ alkyl ester (Cetiol® SN), 2-ethylhexyl palmitate (Cegesoft® 24), stearic acid 2-ethylhexyl ester (Cetiol® 868), cetyl oleate, glycerin tricaprylate, cocofatty alcohol caprinate/-caprylate (Cetiol® LC), n-butyl stearate, oleyl erucate (Cetiol® J 600), isopropyl palmitate (Rilanit® IPP), oleyl oleate (Cetiol®), lauric acid hexyl ester (Cetiol® A), di-n-butyl adipate (Cetiol® B), myristyl myristate (Cetiol® MM), cetearyl isononanoate (Cetiol® SN), oleic acid decyl ester (Cetiol® V) are inventively particularly preferred.
dicarboxylic acid esters such as di-n-butyl adipate, di-(2-ethylhexyl) adipate, di-(2-ethylhexyl) succinate and di-isotridecyl acetate as well as diol esters such as ethylene glycol dioleate, ethylene glycol di-isotridecanoate, propylene glycol di(2-ethylhexanoate), propylene glycol di-isostearate, propylene glycol di-pelargonate, butane diol di-isostearate, neopentyl glycol dicaprylate;
symmetrical, unsymmetrical or cyclic esters of carbon dioxide with fatty alcohols, e.g. glycerin carbonate or dicaprylyl carbonate (Cetiol® CC);
trifatty acid esters of saturated and/or unsaturated linear and/or branched fatty acids with glycerine;
fatty acid partial glycerides, under which are understood monoglycerides, diglycerides and their industrial mixtures. When using industrial products, minor amounts of triglycerides may still be contained as a result of the production process. The partial glycerides preferably comply with the Formula (D4-I)—

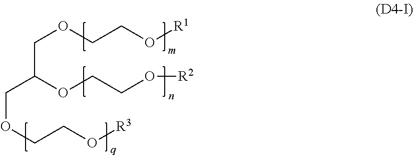

wherein R¹, R² and R³ are each independently hydrogen or a linear or branched, saturated and/or unsaturated acyl group containing 6 to 22 carbon atoms, preferably 12 to 18 carbon atoms, with the proviso that at least one of these groups is an acyl group and at least one of these groups is hydrogen. The sum of (m+n+q) is 0 or numbers from 1 to 100, preferably 0 or 5 to 25. Preferably, R¹ is an acyl group, R² and R³ are hydrogen, and the sum of (m+n+q) is 0. Typical examples include mono- and/or diglycerides based on caproic acid, caprylic acid, 2-ethylhexanoic acid, capric acid, lauric acid, isotridecanoic acid, myristic acid, palmitic acid, palmitoleic acid, stearic acid, isostearic acid, oleic acid, elaidic acid, petroselic acid, linoleic acid, linolenic acid, elaeostearic acid, arachidonic acid, gadoleic acid, behenic acid and erucic acid as well as their industrial mixtures. Oleic acid monoglycerides are preferably employed.

In a most preferred embodiment, the compositions additionally comprise squalene.

The amount of natural and synthetic cosmetic oil bodies added to compositions according to the invention is usually 0.1 to 30 wt. %, based on total composition, preferably 0.1 to 20 wt. % and particularly 0.1 to 15 wt. %. The total amount of oil and fat components in the inventive compositions is normally 0.5-75 wt. %, based on total composition. Quantities of 0.5 to 35 wt. % are inventively preferred.

Another inventive synergistic active principle in compositions according to the invention with the active principle complex are protein hydrolyzates and/or their derivatives (P). According to the invention, “protein hydrolyzates” refers to total hydrolyzates as well as individual amino acids and their derivatives as well as mixtures of different amino acids. Furthermore, according to the invention, polymers built up from amino acids and amino acid derivatives are understood to be included in the term protein hydrolyzates. The latter include polyalanine, polyasparagine, polyserine, etc. Additional examples of usable compounds according to the invention are L-alanyl-L-proline, polyglycine, glycyl-L-glutamine or D/L-methionine-S-methyl sulfonium chloride. Of course, β-amino acids and their derivatives, like β-alanine, anthranilic acid or hippuric acid, can also be inventively employed. The molecular weight of protein hydrolyzates utilizable according to the invention ranges from about 75, the molecular weight of glycine, to about 200,000; preferably the molecular weight is about 75 to about 50,000 and quite particularly preferably about 75 to about 20,000 Dalton.

According to the invention, the added protein hydrolyzates can be of vegetal as well as animal, marine or synthetic origin.

Animal protein hydrolyzates include elastin, collagen, keratin, silk protein, and milk albumin protein hydrolyzates, which can also be present in the form of their salts. Such products are marketed, for example, under the trade names Dehylan® (Cognis), Promois® (Interorgana), Collapuron® (Cognis), Nutrilan® (Cognis), Gelita-Sol® (Deutsche Gelatine Fabriken Stoess & Co), Lexein® (Inolex) and Kerasol® (Croda).

Furthermore, inventively preferred vegetal protein hydrolyzates include soya, almond, pea, potato, moringa, silk and wheat protein hydrolyzates. Such products are available, for example, under the trade names Gluadin® (Cognis), DiaMin® (Diamalt), Lexein® (Inolex) Hydrosoy® (Croda), Hydrolupin® (Croda), Hydrosesame® (Croda), Hydrotritium® (Croda) and Crotein® (Croda).

Additional inventively preferred protein hydrolyzates are of marine origin. These include collagen hydrolyzates of fish or algae as well as protein hydrolyzates of mussels or pearl extracts. Examples of pearl extracts according to the invention are the commercial products Pearl Protein Extract BG® or Crodarom® Pearl.

The compositions comprise protein hydrolyzates in concentrations of 0.001 wt. % to 20 wt. %, preferably 0.05 wt. % to 15 wt. % and quite particularly preferably in amounts of 0.05 wt. % to 5 wt. %, based on total weight of the composition.

Moreover, the action of compositions according to the invention can be augmented by a 2-pyrrolidinone-5-carboxylic acid and its derivatives (J). Accordingly, a further subject matter of the invention is the use of derivatives of 2-pyrrolidinone-5-carboxylic acid. Sodium, potassium, calcium, magnesium or ammonium salts are preferred, wherein the ammonium ion carries one to three C₁ to C₄ alkyl groups besides hydrogen. The sodium salt is quite particularly preferred. The quantities employed in the inventive compositions preferably range from 0.05 to 10 wt. %, based on total composition, particularly preferably 0.1 to 5 wt. %, and particularly 0.1 to 3 wt. %.

Vitamins, provitamins or vitamin precursors are a further preferred group of ingredients of the inventive compositions with the inventive active principle complex. Here, vitamins, provitamins and vitamin precursors assigned to the groups A, B, C, E, F and H are particularly preferred.

The group of substances designated as vitamin A includes retinol (vitamin A1) as well as 3,4-didehydroretinol (vitamin A2). β-Carotene is the provitamin of retinol. Examples of suitable vitamin A components according to the invention are vitamin A acid and its esters, vitamin A aldehyde and vitamin A alcohol as well as its esters such as the palmitate and acetate. Compositions according to the invention preferably comprise vitamin A components in amounts of 0.05 to 1 wt. %, based on total preparation.

In addition to the pantolactone and pantothenic acid and/or their derivatives described above as particularly preferably utilizable, inventively utilizable active substances of the vitamin B group or vitamin B complex include inter alia—
Vitamin B₁ (Thiamine)
Vitamin B₂ (Riboflavin)
Vitamin B₃. The compounds nicotinic acid and nicotinamide (niacinamide) are often included under this designation. According to the invention, nicotinamide is preferred and is comprised in the compositions used according to the invention in amounts of 0.05 to 1 wt. % based on total composition.
Vitamin B₆ (pyridoxine as well as pyridoxamine and pyridoxal).

Vitamin C (ascorbic acid). Vitamin C is preferably added to the compositions according to the invention in amounts of 0.1 to 3 wt. %, based on the total composition. Its use in the form of the palmitate ester, glucosides or phosphates can be preferred. The use in combination with tocopherols can also be preferred.

Vitamin E (Tocopherols, especially α-tocopherol). Tocopherol and its derivatives, among which are particularly included the esters such as the acetate, the nicotinate, the phosphate and the succinate, are used in the compositions according to the invention preferably comprised in amounts of 0.05-1 wt. %, based on the total composition.

Vitamin F. The term “vitamin F” usually refers to essential fatty acids, particularly linoleic acid, linolenic acid and arachidonic acid.

Vitamin H. The compound (3aS,4S,6aR)-2-oxohexahydrothienol[3,4-d]imidazole-4-valeric acid denotes Vitamin H, commonly known as biotin. Compositions according to the invention preferably comprise biotin in amounts of 0.0001 to 1.0 wt. %, particularly 0.001 to 0.01 wt. %. Compositions according to the invention preferably comprise vitamins, provitamins and vitamin precursors from the groups A, B, E and H. Pyridoxine and its derivatives as well as nicotinamide and biotin are especially preferred.

In a preferred embodiment, the inventive compositions can further comprise antimicrobial compounds. Suitable antimicrobial compounds include cationic surface active substances such as cetyltrimethylammonium bromide, benzethonium chloride, cetylpyridinium chloride or the N,N,N-tris-(2-hydroxyethyl)-N′-octadecyl-1,3-diaminopropane dihydrofluoride, known as amine fluoride. Antimicrobially active biguanide compounds are also highly suitable, such as polyhexamethylene biguanide (Vantocil® IB, ICI) or the 1,1′-hexamethylene bis-(4-chlorphenyl)biguanide (“Chlorhexidin”) in the form of a water-soluble, compatible salt, for example, in the form of the acetate or gluconate. The antimicrobial 5-aminohexahydropyrimidines, for example, 1,3-bis-(2-ethylhexyl)-5-methyl-5-aminohexahydropyrimidine (“Hexetidin”), are also suitable and preferred. Further preferred suitable antimicrobial active substances are the non-cationic, phenolic, antimicrobial substances, particularly halogenated phenols and diphenyl ethers. Particularly suitable antimicrobial compounds of this type include 6,6′-methylene bis-(2-bromo-4-chlorophenol) (“Bromchlorophen”) and 2,4,4′-trichloro-2′-hydroxydiphenyl ether (“Triclosan”).

Further suitable antimicrobial substances include p-hydroxybenzoic acid esters and sesquiterpene alcohols such as bisabolol, farnesol, santalol or nerolidol. Finally, additional advantages result from use of vegetal extracts (L) in compositions according to the invention. According to the invention, extracts from green tea, oak bark, stinging nettle, hamamelis, hops, henna, camomile, burdock root, field horsetail, hawthorn, linden flowers, almonds, aloe vera, spruce needles, horse chestnut, sandal wood, juniper, coconut, mango, apricot, lime, wheat, kiwi, melon, orange, grapefruit, sage, rosemary, birch, malva, valeriana, lady's smock, wild thyme, yarrow, thyme, lemon balm, rest-harrow, coltsfoot, marshmallow (althaea), meristem, ginseng, coffee, cocoa, açai palm, goji berry, moringa, common grape vine (Vitis vinifera), Aegle marmelos (bilwa), Cyperus Rotundus (Nagar Motha), Emblica Officinalis (Amalki), Morida Citrifolia (Ashyuka), Tinospora cordifolia (Guduchi), Santalum album, (Chandana), Crocus Sativus (Kumkuma), Cinnamonum Zeylanicum and Nelumbo Nucifera (Kamala) Echinacea and ginger are preferred. According to the invention, plant extracts can be used in pure and in diluted form. When they are used in diluted form, they normally comprise about 2 to 80 wt. % active substance and the solvent is the extracting composition or mixture of extracting compositions used for their preparation.

In addition, it can be preferred to employ mixtures of a plurality, in particular at least two or more, different plant extracts in compositions according to the invention. The inventive compositions can include purine and/or derivative(s) of purine as additional ingredients. Purine and/or purine derivatives are often isolated from natural products, but can also be obtained synthetically by numerous routes.

Preferred compositions according to the invention include purine and/or purine derivatives in narrow quantitative ranges. Inventively preferred cosmetics comprise, based on their weight, 0.001 to 2.5 wt. %, preferably 0.0025 to 1 wt. %, particularly preferably 0.005 to 0.5 wt. % and particularly 0.01 to 0.1 wt. % purine and/or purine derivative(s).

Inventively preferred cosmetics comprise purine and/or purine derivative(s) of formula (I) or (II)—

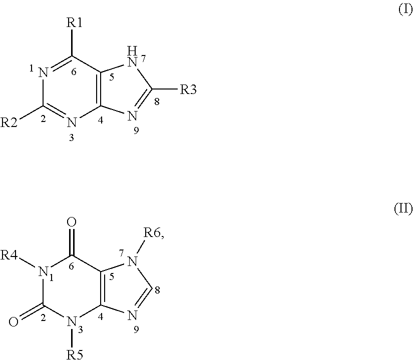

wherein the groups R1, R2 and R3 of formula (I) are each independently —H, —OH, —NH₂, or —SH, and the groups R4, R5 and R6 of formula (II) are each independently —H, —CH₃ or —CH₂-CH₃, wherein the following substances according to formula (I) are preferred:
Purine (R1=R2=R3=H)
Guanine (R1=OH, R2=NH₂, R3=H)
Ureic acid (R1=R2=R3=OH)
Hypoxanthine (R1=OH, R2=R3=H)
6-Purinthiol (R1=SH, R2=R3=H)
6-Thioguanine (R1=SH, R2=NH₂, R3=H)
Xanthine (R1=R2=OH, R3=H)
   and the following compounds according to formula (II) are preferred:
Caffeine (R4=R5=R6=CH₃)
Theobromine (R4=H, R5=R6=CH₃)
Theophylline (R4=R5=CH₃, R6=H)

Caffeine has proven especially effective in hair cosmetic formulations and can be used, for example, in shampoos, conditioners, hair tonics and/or lotions preferably in amounts of 0.005 to 0.25 wt. %, more preferably 0.01 to 0.1 wt. % and especially 0.01 to 0.05 wt. %, based on total weight of the composition).

In addition, it can prove advantageous when the inventive compositions include penetration aids and/or swelling agents (M). These include urea and urea derivatives, guanidine and its derivatives, arginine and its derivatives, water glass, imidazole and its derivatives, histidine and its derivatives, benzyl alcohol, glycerine, glycol and glycol ethers, propylene glycol and propylene glycol ethers, for example propylene glycol monoethyl ether, carbonates, hydrogen carbonates, diols and trials, and particularly 1,2-diols and 1,3-diols such as for example 1,2-propane diol, 1,2-pentane diol, 1,2-hexane diol, 1,2-dodecane diol, 1,3-propane diol, 1,6-hexane diol, 1,5-pentane diol, 1,4-butane diol. A particularly preferred group of swelling agents can be hydantoins. Hydantoins display several desirable advantageous effects in cosmetic compositions. One of these effects is their ability to favorably influence the swelling of the skin and the hair. Consequently, compositions according to the invention preferably comprise 0.01 to 5 wt. % hydantoin or at least one hydantoin derivative. Hydantoin derivatives are particularly preferably inventively employed, 5-ureido hydantoin being particularly preferred. Independently of whether hydantoin or hydantoin derivative(s) is/are employed, added quantities of 0.02 to 2.5 wt. % are quite particularly preferred, 0.05 to 1.5 wt. % are further preferred, 0.075 to 1 wt. % and especially 0.1 to 0.25 wt. % are most preferred, based on total weight of the composition.

Furthermore, in order to achieve a specific effect such as an anti-dandruff effect or actions against acne, cosmetic compositions according to the invention can comprise pharmacologically active substances. Exemplary pharmacologically active substances are corticosteroids, β-blockers, estrogens, phytoestrogens, cyproterone acetate, vasodilatory active substances such as Diazoxid, Nifedipin and Minoxidil, acetylsalicylic acid or salicylic acid. These substances can be present in amounts of 0.01 to 10 wt. %, based on total composition.

Perfumes are generally added to the total composition in a quantity of 0.05 to 5 wt. %, preferably in a quantity of 0.1 to 2.5 wt. % and particularly in a quantity of 0.2 to 1.5 wt. %, based on total composition.

Accordingly, in a preferred embodiment the compositions according to the invention comprise a bittering agent in order to prevent swallowing or accidental ingestion. Bittering agents having water-solubility at 20° C. of at least 5 g/l are inventively preferred.

Quaternary ammonium compounds that comprise an aromatic group in both the cation and the anion are inventively highly suitable as bittering agents. A compound of this type is benzyldiethyl((2,6-xylylcarbamoyl)methyl)ammonium benzoate, commercially available for example under the trade names Bitrex® and Indigestin®. This compound is also known by the name Denatonium Benzoate.

The bittering agent is comprised in the compositions according to the invention in quantities of 0.0005 to 0.1 wt. %, based on the molded article. Quantities of 0.001 to 0.05 wt. % are particularly preferred.

In the context of the invention, it is inventively quite particularly preferred to use the so-called food acids as short chain carboxylic acids. The compositions comprise these active substances according to the invention in concentrations of 0.01 wt. % to 20 wt. %, preferably 0.05 wt. % up to 15 wt. % and quite particularly preferably in amounts of 0.1 wt. % up to 5 wt. %.

For example, polyols according to the invention include sorbitol, inositol, mannitol, tetritols, pentitols, hexitols, threitol, erythritol, adonitol, arabitol, xylitol, dulcitol, erythrose, threose, arabinose, ribose, xylose, lyxose, glucose, galactose, mannose, allose, altrose, gulose, idose, talose, fructose, sorbose, psicose, tegatose, desoxyribose, glucosamine, galactosamine, rhamnose, digitoxose, thioglucose, saccharose, lactose, trehalose, maltose, cellobiose, melibiose, gestiobiose, rutinose, raffinose as well as cellotriose.

Preferred polyhydroxy compounds include sorbitol, inositol, mannitol, threitol, erythritol, erythrose, threose, arabinose, ribose, xylose, glucose, galactose, mannose, allose, fructose, sorbose, desoxyribose, glucosamine, galactosamine, saccharose, lactose, trehalose, maltose and cellobiose. Glucose, galactose, mannose, fructose, desoxyribose, glucosamine, saccharose, lactose, maltose and cellobiose are particularly preferably used. However, the use of glucose, galactose, mannose, fructose, saccharose, lactose, maltose or cellobiose is quite particularly preferred.

Quite particularly preferred polyols of the present invention contain 2 to 12 carbon atoms in the molecular structure. These polyols can be straight chain, branched, cyclic and/or unsaturated. The hydroxyl groups are quite particularly preferably terminally adjacent or terminal and separated from one another by the remainder of the chain. The following may be cited as examples of these polyols: glycol, polyethylene glycol up to a molecular weight of 1000 dalton, neopentyl glycol, partial ethers of glycerin with a molecular weight of up to 1000 Dalton, 1,2-propane diol, 1,3-propane diol, glycerin, 1,2-butane diol, 1,3-butane diol, 1,4-butane diol, 1,2,3-butane triol, 1,2,4-butane triol, pentane diols, for example 1,2-pentane diol, 1,5-pentane diol, hexane diols, 1,2-hexane dial, 1,6-hexane diol, 1,2,6-hexane triol, 1,4-cyclohexane diol, 1,2-cyclohexane diol, heptane diols, 1,2-heptane diol, 1,7-heptane diol, octane diols, 1,2-octane diol, 1,8-octane diol, 2-ethyl-1,3-hexane diol, octadienols, decadienols, dodecane diols, 1,2-dodecane diol, 1,12-dodecane diol, 1,12-dodecane diol with 10 moles EO, dodecadienols. Glycerin is quite particularly preferred.

Independently of the type of the added polyhydroxy compound that contains at least 2 OH groups, preferred inventive compositions comprise, based on weight of the composition, 0.01 to 5 wt. %, preferably 0.05 to 4 wt. %, particularly preferably 0.05 to 3.5 wt. % and particularly 0.1 to 2.5 wt. % of polyhydroxy compound(s).

According to the invention, it is also possible to employ a mixture of a plurality of polyhydroxy compounds.

Polyhydroxy compounds according to the invention are comprised in the compositions in concentrations of 0.01 wt. % to 50 wt. %, preferably 0.05 wt. % up to 35 wt. % and quite particularly preferably in amounts of 0.1 wt. % up to 20 wt. %.

In a further particularly preferred embodiment, compositions according to the invention additionally comprise at least one UV light filter. Compositions according to the invention preferably comprise UV-A and/or UV-B filters.

UV-B filters can be oil-soluble or water-soluble. As oil-soluble substances, the following may be cited—
3-benzylidene camphor and its derivatives (e.g., 3-(4-methylbenzylidene)camphor);
4-aminobenzoic acid derivatives, preferably 2-ethylhexyl 4-(dimethylamino)benzoate, 2-octyl 4-(dimethylamino)benzoate and amyl 4-(dimethylamino)benzoate;
esters of cinnamic acid, preferably 2-ethylhexyl 4-methoxycinnamate, propyl 4-methoxycinnamate, isoamyl 4-methoxycinnamate, 2-ethylhexyl 2-cyano-3,3-phenylcinnamate (Octocrylene);
esters of salicylic acid, preferably 2-ethylhexyl salicylate, 4-isopropylbenzyl salicylate, homomenthyl salicylate;
derivatives of benzophenone, preferably 2-hydroxy-4-methoxybenzophenone, 2-hydroxy-4-methoxy-4′-methylbenzophenone, 2,2′-dihydroxy-4-methoxybenzophenone;
esters of benzalmalonic acid, preferably di-2-ethylhexyl 4-methoxybenzmalonate;
triazine derivatives, such as 2,4,6-trianilino-(p-carbo-2′-ethyl-1′-hexyloxy)-1,3,5-triazine and octyl triazone; and
propane-1,3-dione, such as 1-(4-tert.butylphenyl)-3-(4′-methoxyphenyl)propane-1,3-dione.

Water-soluble substances include—
2-phenylbenzimidazole-5-sulfonic acid and its alkali metal, alkaline earth metal, ammonium, alkylammonium, alkanolammonium and glucammonium salts;
sulfonic acid derivatives of benzophenones, preferably 2-hydroxy-4-methoxybenzophenone-5-sulfonic acid and its salts; and
sulfonic acid derivatives of 3-benzylidenecamphor, such as 4-(2-oxo-3-bornylidenemethyl)benzene sulfonic acid and 2-methyl-5-(2-oxo-3-bornylidene)sulfonic acid and their salts.

Typical UV-A filters include derivatives of benzoylmethane such as 1-(4′-tert.butylphenyl)-3-(4′-methoxyphenyl)propane-1,3-dione or 1-phenyl-3-(4′-isopropylphenyl)-propane-1,3-dione. Naturally, the UV-A and UV-B filters can also be added as mixtures. Beside the cited soluble materials, insoluble, light protective pigments, namely finely dispersed metal oxides or salts can also be considered for this purpose, such as titanium dioxide, zinc oxide, iron oxide, aluminum oxide, cerium oxide, zirconium oxide, silicates (talc), barium sulfate and zinc stearate. Here, the particles should have a mean diameter of about 100 nm or less, preferably from about 5 to about 50 nm and especially from about 15 to about 30 nm. They can be spherical; however elliptical or other non-spherical shaped particles can also be used. As well as both above-cited groups of primary light protective materials, secondary light protective agents of the antioxidant type can also be used, which interrupt photochemical chain reactions that are propagated when the UV-radiation penetrates the skin. Typical examples of these are superoxide dismutase, tocopherols (vitamin E) and ascorbic acid (vitamin C).

Additional optional ingredients that can be used together in cosmetic compositions include preservatives. Generally the following substances and their mixtures are used—
aromatic alcohols such as phenoxyethanol, benzyl alcohol, phenethyl alcohol, phenoxyisopropanol;
aldehydes such as formaldehyde solution and paraformaldehyde, glutaraldehyde;
parabens such as methyl paraben, ethyl paraben, propyl paraben, butyl paraben, isobutyl paraben;
1,2-alkane diols containing 5 to 22 carbon atoms in the carbon chain, such as 1,2-pentane diol, 1,2-hexane diol, 1,2-heptane diol, 1,2-decane diol, 1,2-dodecane dial, 1,2-hexadecane diol;
formaldehyde-eliminating compounds, such as DMDM hydantoin, diazolidinyl urea;
halogenated compounds such as isothiazolinones, such as methylchloroisothiazolinone/methylisothiazolinones, Triclosan, Triclocarban, iodopropynylbutyl carbamate, 5-bromo-5-nitro-1,3-dioxane, chlorhexidine digluconate and chlorhexidine acetate, 2-bromo-2-nitropropane-1,3-diol, methyldibromoglutaronitrile;
inorganic compounds, such as sulfites, boric acid and borates, bisulfites;
cationic substances such as Quaternium-15, Benzalkonium chloride, Benzethonium chloride, polyaminopropyl biguanide;
organic acids and their physiologically compatible salts such as citric acid, lactic acid, acetic acid, benzoic acid, sorbic acid, salicylic acid, dehydroacetic acid;
active substances with additional effects such as zinc pyrithion, piroctonolamine;
antioxidants such as BHT (butylated hydroxytoluene), BHA (butylated hydroxyanisole), propyl gallate, t-butylhydroquinone; and
complexants such as EDTA and its derivatives, HEDTA and its derivatives, etidronic acid and its salts;
   as well as mixtures of the substances listed above.

Preservatives are used in quantities of 0 to 5 wt. %, preferably 0 to 2 wt. %, particularly preferably 0 to 1 wt. % and quite particularly preferably 0 to 0.8 wt. %, based on total amount of the composition.

Exemplary additional active substances, adjuvants and additives which can be present in the cosmetic compositions include—
perfume oils, dimethyl isosorbitol and cyclodextrins;
dyestuffs to color the composition;
anti-dandruff active materials such as Piroctone Olamine, zinc Omadine and Climbazole;
cholesterol;
chelating agents like EDTA, NTA, β-alanine diacetic acid and phosphonic acids;
opacifiers such as latex, styrene/PVP copolymers and styrene/acrylamide copolymers;
pearlizing compositions such as ethylene glycol mono- and distearate as well as PEG-3-distearate;
pigments;
stabilizers for hydrogen peroxide and other oxidizing agents;
propellants such as propane-butane mixtures, N₂O, dimethyl ether, CO₂ and air; and
antioxidants.

The pH of compositions according to the invention is preferably from 2 to 12. The pH of cosmetic compositions according to the invention is adjusted by using the above listed carboxylic acids or mineral acids (e.g., phosphoric acid, sulfuric acid, etc.).

Inorganic compounds, preferably hydroxides or ammonia as well as organic bases, preferably amines, including in particular alkanolamines, can be employed as alkalizing agents.

Preferred compositions according to the invention contain in a cosmetic carrier, in addition to a cloudberry fruit extract, one of the following combinations of active substances (i) to (xx)—
(i) at least one color-changing component and at least one silicone;
(ii) at least one color-changing component and at least one cationic surfactant;
(iii) at least one color-changing component and at least one cationic polymer;
(iv) at least one color-changing component and at least one UV-absorbent;
(v) at least one developer component (and optionally a coupler component) and at least one silicone;
(vi) at least one developer component (and optionally a coupler component) and at least one cationic surfactant;
(vii) at least one developer component (and optionally a coupler component) and at least one cationic polymer;
(viii) at least one developer component (and optionally a coupler component) and at least one UV-absorbent;
(ix) at least one bleach booster and at least one silicone;
(x) at least one bleach booster and at least one cationic surfactant;
(xi) at least one bleach booster and at least one cationic polymer;
(xii) at least one bleach booster and at least one UV-absorbent;
(xiii) pantolactone and at least one cationic surfactant;
(xiv) pantolactone and at least one cationic polymer;
(xv) pantolactone and at least one silicone;
(xvi) pantolactone and at least one UV-absorbent;
(xvii) ectoine and at least one cationic surfactant;
(xviii) ectoine and at least one cationic polymer;
(xix) ectoine and at least one silicone; and
(xx) ectoine and at least one UV-absorbent.

Preferred compositions according to the invention contain in a cosmetic carrier, in addition to the combination of (a) at least one oxidizing agent (preferably hydrogen peroxide) and (b) a cloudberry fruit extract, one of the following combinations of active substances (i) to (xx)—
(i) at least one color-changing component and at least one silicone;
(ii) at least one color-changing component and at least one cationic surfactant;
(iii) at least one color-changing component and at least one cationic polymer;
(iv) at least one color-changing component and at least one UV-absorbent;
(v) at least one developer component (and optionally a coupler component) and at least one silicone;
(vi) at least one developer component (and optionally a coupler component) and at least one cationic surfactant;
(vii) at least one developer component (and optionally a coupler component) and at least one cationic polymer;
(viii) at least one developer component (and optionally a coupler component) and at least one UV-absorbent;
(ix) at least one bleach booster and at least one silicone;
(x) at least one bleach booster and at least one cationic surfactant;
(xi) at least one bleach booster and at least one cationic polymer;
(xii) at least one bleach booster and at least one UV-absorbent;
(xiii) pantolactone and at least one cationic surfactant;
(xiv) pantolactone and at least one cationic polymer;
(xv) pantolactone and at least one silicone;
(xvi) pantolactone and at least one UV-absorbent;
(xvii) ectoine and at least one cationic surfactant;
(xviii) ectoine and at least one cationic polymer;
(xix) ectoine and at least one silicone; and
(xx) ectoine and at least one UV-absorbent.

Preferred compositions according to the invention contain in a cosmetic carrier, in addition to the combination of (a) at least one oxidizing agent (preferably hydrogen peroxide) and (b) a cloudberry fruit extract, one of the following combinations of active substances (i) to (xv)—
(i) at least one color-changing component and at least one fat;
(ii) at least one color-changing component and at least one C₁₀ to C₂₂ fatty alcohol;
(iii) at least one color-changing component and at least one C₁₀ to C₂₂ fatty acid;
(iv) at least one color-changing component and at least one naturally occurring or synthetic wax;
(v) at least one color-changing component and at least one naturally occurring or synthetic oil component;
(vi) at least one developer component (and optionally a coupler component) and at least one fat;
(vii) at least one developer component (and optionally a coupler component) and at least one C₁₀ to C₂₂ fatty alcohol;
(viii) at least one developer component (and optionally a coupler component) and at least one C₁₀ to C₂₂ fatty acid;
(ix) at least one developer component (and optionally a coupler component) and at least one naturally occurring or synthetic wax;
(x) at least one developer component (and optionally a coupler component) and at least one naturally occurring or synthetic oil component;
(xi) at least one bleach booster and at least one fat;
(xii) at least one bleach booster and at least one C₁₀ to C₂₂ fatty alcohol;
(xiii) at least one bleach booster and at least one C₁₀ to C₂₂ fatty acid;
(xiv) at least one bleach booster and at least one naturally occurring or synthetic wax; and
(xv) at least one bleach booster and at least one naturally occurring or synthetic oil component.

Preferred compositions according to the invention contain in a cosmetic carrier, in addition to the combination of (a) at least one oxidizing agent (preferably hydrogen peroxide) and (b) a cloudberry fruit extract, one of the following combinations of active substances (i) to (xv)—
(i) at least one color-changing component and ammonia;
(ii) at least one color-changing component and at least one alkanolamine;
(iii) at least one color-changing component and 2-aminoethanol;
(iv) at least one color-changing component and ammonia and 2-aminoethanol;
(v) at least one color-changing component and 2-aminoethanol and L-arginine;
(vi) at least one developer component (and optionally a coupler component) and ammonia;
(vii) at least one developer component (and optionally a coupler component) and at least one alkanolamine;
(viii) at least one developer component (and optionally a coupler component) and 2-aminoethanol;
(ix) at least one developer component (and optionally a coupler component) and ammonia and 2-aminoethanol;
(x) at least one developer component (and optionally a coupler component) and 2-aminoethanol and L-arginine;
(xi) at least one bleach booster and ammonia;
(xii) at least one bleach booster and at least one alkanolamine;
(xiii) at least one bleach booster and 2-aminoethanol;
(xiv) at least one bleach booster and ammonia and 2-aminoethanol; and
(xv) at least one bleach booster and 2-aminoethanol and L-arginine.

A second subject matter of the invention is the use of a cloudberry fruit extract in compositions according to the invention to increase the moisture of keratin fibers.

A third subject matter of the invention is the use of a cloudberry fruit extract in compositions according to the invention to repair and restructure keratin fibers.

A fourth subject matter of the invention is the use of a cloudberry fruit extract in compositions according to the invention to protect keratin fibers from oxidizing agents and UV radiation as well as from other oxidative damage to keratin fibers.

A fifth subject matter of the invention is the use of a cloudberry fruit extract in compositions according to the invention to improve the color fastness and the color intensity of the dyed keratin fibers.

A sixth subject matter of the invention is a method for treating keratin fibers in which a cosmetic composition, comprising a cloudberry fruit extract in a cosmetic carrier, is applied and optionally rinsed out again after a contact time.

The contact time of the cloudberry fruit extract is preferably a few seconds to 100 minutes, particularly preferably a few seconds to 45 minutes and quite particularly preferably 1 to 30 minutes.

A preferred embodiment of the present invention is a method for oxidative hair treatment, in which an oxidative cosmetic composition comprising at least one oxidizing agent in a cosmetic carrier is applied onto the hair and is rinsed after a contact time, wherein prior to the application of the oxidative cosmetic composition (preferably nearly immediately prior to), a cosmetic composition comprising a cloudberry extract in a cosmetic carrier is applied and optionally rinsed out again after a contact time.

It is once again inventively preferred to use the inventive method in the context of an oxidative hair dyeing, oxidative hair bleaching or setting in the context of a permanent set. In this case it is preferred to apply the cloudberry fruit extract together with the oxidizing agent on the hair.

In a preferred embodiment of the inventive method as an oxidative hair dyeing method, the cloudberry fruit extract as an ingredient of a cosmetic composition comprising at least one oxidizing agent and at least one oxidation dye precursor in a cosmetic carrier is preferably applied in one step. The oxidative hair dye compositions of this embodiment are preferably two-component compositions. The first component comprises a cloudberry fruit extract in a cosmetic carrier and at least one oxidation dye precursor. The second component comprises at least one oxidizing agent in a cosmetic carrier. These components are preferably packaged separately from one another, each in a compartment, and provided together in a kit. Both components are blended together shortly before use.

In the embodiment of the inventive method as an oxidative hair bleaching method, the cloudberry fruit extract as an ingredient of a cosmetic composition additionally comprising at least one oxidizing agent and at least one oxidation dye precursor in a cosmetic carrier is preferably applied in one step.

Oxidative hair bleaching compositions of this embodiment are preferably two- or three-component compositions. The first component comprises a cloudberry fruit extract in a cosmetic carrier and optionally at least one bleach booster. The bleach booster can also be packaged separately from the cloudberry fruit extract, for example, in powder form, as an anhydrous paste or anhydrous oil. A three-component composition is obtained in this manner. The last component comprises at least one oxidizing agent in a cosmetic carrier. All components are preferably packaged separately from one another, each in a compartment, and provided together in a kit. All components are blended together shortly before use. The previously described multi-component compositions for carrying out the inventive method can be provided in one kit. The kit can include either at least one container containing the composition of the second subject matter of the invention, or can include at least two containers, wherein a first container comprises an oxidative cosmetic composition comprising at least one oxidizing agent in a cosmetic carrier, a second container comprises a cosmetic composition comprising a cloudberry fruit extract in a cosmetic carrier, and optionally a third container that comprises at least one bleach booster embedded in a cosmetic carrier. All containers can also be chambers of a multi-chamber container.

The following examples are intended to illustrate the subject matter of the invention in more detail, without limiting it in any way.

### EXEMPLARY FORMULATIONS

A. Hair Rinse—

| Raw material/Ingredient | Content in wt. % |
|---|---|
| Stenol 1618¹ | 3.0 |
| Genamin KDPM² | 1.0 |
| Rheocare Ultragel³ | 2.0 |
| Pantolactone | 0.2 |
| Fruitliquid Cloudberry | 0.5 |
| Methyl paraben | 0.2 |
| Perfume | 0.3 |
| 2-Phenoxyethanol | 0.4 |
| Water | ad 100 |

¹Stenol ® 16/18 C₁₆-C₁₈ fatty alcohol (INCI name: Cetearyl alcohol; (Cognis)
²Genamin ® KDMP C₂₀-₂₂ alkyltrimethylammonium chloride (INCI: Behentrimonium Chloride; Clariant)
³Rheocare Ultragel ® 300 2-(trimethylammonio)ethyl methacrylate chloride homopolymer (INCI: Polyquaternium-37; Cognis)
⁴Fruitliquid Cloudberry (INCI: Glycerin, Aqua, Rubus chamaemorus Fruit Extract; Crodarom)

B. Hair Tonic—

| Raw material/Ingredient | Content in wt. % |
|---|---|
| Stenol 1618 | 6.0 |
| Genamin KDPM | 1.5 |
| Rheocare Ultragel | 3.0 |
| Pantolactone | 0.5 |
| Fruitliquid Cloudberry | 1.0 |
| Methyl paraben | 0.2 |
| Perfume | 0.3 |
| 2-Phenoxyethanol | 0.4 |
| Water | ad 100 |

We claim:

1. Cosmetic composition for the treatment of keratin fibers comprising an extract of fruits of Rubus chamaemorus (cloudberry) in a cosmetically acceptable carrier.

2. Cosmetic composition according to claim 1 wherein the cloudberry fruit extract is present in an amount of 0.1 to 5.0 wt. %, based on total weight of the composition.

3. Cosmetic composition according to claim 1 further comprising at least one additional active substance chosen from pantolactone, non-surface active betaines, ubiquinone, ectoine, pantothenic acid, nicotinic acid, their derivatives or physiologically acceptable salts.

4. Cosmetic composition according to claim 3 wherein the additional active substance is pantolactone, carnitine tartrate, coenzyme Q-10, ectoine, pantothenic acid, taurine, panthenol, nicotinamide or mixtures thereof.

5. Cosmetic composition according to claim 3 wherein the weight ratio of the cloudberry fruit extract to the additional active substance is in a range of 1:30 to 50:1.

6. Cosmetic composition according to claim 1 further comprising at least one cyclic or linear polyol comprising 1 to 8 carbon atoms in its chain and at least two hydroxyl groups.

7. Cosmetic composition according to claim 1 further comprising at least one polyol and the at least one polyol is at least glycerin.

8. Cosmetic composition according to claim 1 further comprising at least one skin or hair conditioner chosen from cationic polymers, cationic surfactants, silicones, vegetal oils or mixtures thereof.

9. Cosmetic composition according to claim 1 wherein the composition is in the form of a shampoo, hair tonic, hair rinse, hair foam, hair spray, hair gel or hair dye.

10. Method of increasing the moisture content of keratin fibers comprising applying the composition of claim 1 to keratin fibers.

11. Method of repairing and restructuring keratin fibers comprising applying the composition of claim 1 to keratin fibers.

12. Method of protecting keratin fibers from oxidative and UV damage comprising applying the composition of claim 1 to keratin fibers.

13. Method of improving color fastness and color intensity of dyed keratin fibers comprising applying the composition of claim 1 to keratin fibers.

14. Method of treating keratin fibers comprising applying the composition of claim 1 to keratin fibers and rinsing out the composition after a contact time of one second up to 45 minutes.
